(19) United States
(12) Patent Application Publication
(10) Pub. No.: US 2007/0231312 A1
(43) Pub. Date: Oct. 4, 2007

(54) NUTRITIONAL SUPPLEMENT FOR PREGNANT FEMALES

(76) Inventors: Michael V. Muench, Point Pleasant, NJ (US); Lori L. Muench, Point Pleasant, NJ (US); Steven M. Feld, Loch Arbour (Allenhurst), NJ (US)

Correspondence Address: ARTHUR L. LESSLER, 540 OLD BRIDGE TURNPIKE, SOUTH RIVER, NJ, 08882, US

(21) Appl. No.: 11/552,015

(22) Filed: Oct. 23, 2006

Related U.S. Application Data
(60) Provisional application No. 60/744,232, filed on Apr. 4, 2006.

(51) Int. Cl. A61K 38/43; A61K 31/714; A61K 31/675; A61K 31/525; A61K 31/4415; A61K 31/405; A61K 31/375; A61K 31/415; A61K 31/198; A61K 31/192; A61K 31/616

(52) U.S. Cl. 424/94.1; 424/702; 424/643; 514/52; 514/165; 514/251; 514/89; 514/350; 514/458; 514/763; 514/562; 514/474; 514/419; 514/406; 514/570

(57) ABSTRACT

Preeclampsia and intrauterine growth restriction in a pregnant female mammal are prevented or decreased in severity by administering thereto a combination of a vitamin compound containing B1, Folic Acid (or active form 5-Methyl-Tetrahydrofolate i.e.; Metafolin®), B 6 (Pyridoxine or active form Pyridoxine 5-Phosphate P5P), B 12, Ascorbic Acid, Selenium, Zinc, Co-enzyme Q 10 and N-Acytyl Cysteine, Lycopene, optionally in further combination with Melatonin and/or Vitamin E or/and ASA 81 mg both of later, are cyclooxygenase inhibitors, a PGI.sub.2-mimetic, a thromboxane (TXA.sub.2) inhibitor, a compound possessing TXA.sub.2-agonistic and TXA.sub.2-inhibiting properties, a compound possessing TXA.sub.2-antagonistic and PGI.sub.2-mimetic activities, and a TXA.sub.2 antagonist.

This application claims the benefit of U.S. Provisional Application Ser. No. 60/744,232 filed Apr. 4, 2006 and entitled Nutritional Supplement For Pregnant Females

### BACKGROUND OF THE INVENTION

This invention relates to a method for the treatment and/or prevention of preeclampsia, intrauterine growth restriction, and or complication resulting from oxidative stress during pregnancy with the combination of a vitamins, coenzymes and amino acid substrate or in further combination with vitamin E one or more of a cyclooxygenase inhibitor, a PGI.sub.2-mimetic, a thromboxane (TXA.sub.2) inhibitor, a compound possessing TXA.sub.2-agonistic and TXA.sub.2-inhibiting properties, a compound possessing TXA.sub.2 antagonistic and PGI.sub.2-mimetic activities, and a TXA.sub.2 antagonist, and to pharmaceutical compositions comprising such a combination.

Preeclampsia, toxemia or eclampsia of pregnancy can be a significant health problem during pregnancy, and they are the leading causes of fetal growth restriction, fetal mortality and morbidity, premature birth and maternal mortality. The etiology of the disease is largely unknown and effective therapy is not available. Preeclampsia of pregnancy is characterized by a triad of hypertension, pathological edema and proteinuria. This disease affects 6 to 10% of all pregnancies.

During normal pregnancy, the placenta produces significant quantities of reactive oxygen species and lipid peroxides. In women with dyslipidemia and increased lipid peroxides, the ability of the antioxidant systems to neutralize placental lipid peroxides is exceeded and a pathological state of oxidative stress develops. The placental contribution to increased circulating levels of lipid peroxides may serve as the ‘catalyst’ for increased oxidative stress in preeclampsia which, when eliminated at the time of delivery, results in disease regression.

Women with preeclampsia have increased circulating levels of oxygen free radicals and lipid peroxides. Conversely, women with preeclampsia have decreased levels of circulating antioxidants. This vitamin supplement has been developed specifically to decrease oxidative stress. As a result of an imbalance of oxidant and antioxidant activity involving placental and maternal lipid peroxidation, the multiorgan endothelial dysfunction observed among preeclamptic patients appears to be related to damage caused by unregulated free radial production. Free radical production during lipid peroxidation is central to the induction of disease. Free radicals are molecules that contain 1 or more unpaired eletrons, typified by oxygen-containing hydroxyl radical (HO⁻), superoxide anion radical (O₂⁻), and nitric oxide (NO⁻). Reactive oxygen species (ROS) include free radicals, but also include molecules that do not have unpaired electrons such as hydrogen peroxide (H₂O₂), hypochlorous acid (HOCl), and peroxynitrite anion (ONOO⁻). The lipid hydroperoxides are highly reactive products of lipid peroxidation formed by the action of free radicals on fatty acids or cholesterol in endothelial membranes, damaging it and producing the resultant endothelial cell dysfunction. As early as 1983, Saeed and Mitchell¹ studies the formation of lipoxygenase metabolites by human uterine and intrauterine tissues. Their early observations suggested that uterine and intrauterine tissues were potential sources of lipoxygenase products during pregnancy and parturition, and that aberrant lipoxygenase activities may contribute to the complication of preeclampsia. Recently it has been reported that there is an abnormal turnover in the trophoblastic cells of the placenta in the presence of oxidative stress (low oxygen levels, decreased nutritional support). The abnormal turnover results in the release of free fetal DNA which is thought to be the causative agent for maternal endothelial injury and one of the initiators of the syndrome know as preeclampsia.

Antioxidants are molecules, which scavenge free radicals and block the chain reaction of unregulated oxidation before damage occurs. There are several enzyme systems within the body that scavenge free radicals, including superoxide dismutase (SOD), catalase, glutathione peroxidase (GSHPx), and glutathione reductase. The principle micronutrient (vitamin) antioxidants are vitamins C and E as well as carotenoids such as beta-carotene. Additionally, selenium, a trace metal and cofactor for glutathione peroxidase behaves as an antioxidant and peroxynitrite scavenger when incorporated into selenoproteins. Zinc is an important trace element implicated in scavenging free radicals. Deficient zinc levels decrease cytosolic superoxide dismutase activity resulting in depleted antioxidant potential². These are essential micronutrients and they must be supplied in the diet. Additionally, coenzyme Q, melatonin, and nicotine are important mitochondrial antioxidants.^{3,4,5,6,7,8,9} Together these vitamins, enzymes, and cofactors provide protection from the potentially damaging consequences of reactive oxygen species to endothelial cells.

### Introduction for Vitamins, Trace Elements and Proteins Contain in Nutritional Compound:

### Vitamin B6 and B12

Homozygosity for cytosine 677 thymine (C677T) mutation in methylenetetrahydrofolate reductase (MTHFR) results in decreased synthesis of 5-methyltetrahydrofolate, the primary methyl donor in the conversion of homocysteine to methionine, and the resulting increase in plasma homocysteine concentration is a risk factor for thrombosis¹⁰. The mutation responsible for reduced MTHFR activity and is the most frequent cause of mild hyperhomocysteinemia and can be found in 5%-15% of the population. Homocysteine is an independent risk factor for atherosclerosis, stroke, peripheral vascular disease, and cardiovascular diseases. Homocysteine concentrations are effected by nutrition. A defiency in Folate Acid, B-6, and/or B-12 causes elevation of homocysteine. Homocysteine concentrations are also effected by genetics such as cystathione beta-synthase defiency and C677T MTHFR gene mutation. Hyperhomocysteinemia promotes vascular damage by several mechanisms. Many of the endothelial vascular changes associated with hyperhomocysteinemia can be found in preeclampsia². It has been proven that elevated homocysteine levels are associated with an increased risk for preeclampsia and intrauterine growth restriction. These levels can be decreased with the addition of Folic Acid, B-6 and B-12.

### Vitamin C and Vitamin E.

Vitamin E is hydrophobic in nature and it is located in biological membranes and lipoproteins. It is a chain-breaking antioxidant which means it will disrupt the radical chain reactions of lipid peroxidation. Vitamin C (Ascorbic Acid) is a reducing agent in a number of different reactions. Vitamin C has the potential to reduce cytochromes A and C of the respiratory chain as well as molecular oxygen. Chappell et al¹¹ conducted a blind, randomized, placebo-controlled trial of antioxidant vitamins aimed at preventing the development of preeclampsia. Women with a history of preeclampsia or with abnormal uterine artery Dopplers in the second trimester (18-22 weeks gestation) were identified as being high risk for the development of preeclampsia. Those identified as high risk were randomized to receive vitamin C (1000 μg. per day) and vitamin E (400 mIU per day) or placebo. Women initially identified as high risk by abnormal second-trimester uterine artery Doppler waveforms analyzed by intent to treat demonstrated nearly a 60% reduction in the occurrence of preeclampsia from 17% in the placebo group to 8% in the treat group. This study suggests a 76% reduction in the rate of preeclampsia among these at-risk patients. Women with preeclampsia who finished the trial demonstrated a reduction in endothelial cell activation compared with normotensive women who finished the trial. Women who received antioxidant supplementation demonstrated biochemical improvements in measures of oxidative stress.

### Selenium

Selenium is important in the role of glutathione peroxidase pathway to remove damaged cellular peroxidases, hence antioxidant activity. Serum Selenium concentrations are reduced in women with preeclampsia compared with normotensive pregnant controls¹². Rayman et al¹³ measured concentrations of Selenium in toenail clippings of women who developed preeclampsia and matched controls. Toenail clippings are a reliable measurement of Selenium status in the previous 3 to 12 months, suggesting that decreased Selenium occurs before the onset of clinical disease in women destined to develop preeclampsia. A group from Beijing¹⁴ randomized 52 women at high risk for developing preeclampsia to receive 1000 μg. per day of liquid Selenium or placebo for 6 to 8 weeks during late gestation. There was a decreased incidence of pregnancy-induced hypertension from 22.7% to 7.7% in the treatment group.

### Co-enzyme Q 10

CoQ10 (2,3-dimethoxy-5-methylbenzoquinone) is chemically classified as a fat-soluble quinone ring attached to 10 isoprene side units, structurally similar to vitamin K. In humans, CoQ10 is found in relatively higher concentrations in cells with high energy requirements. Co-enzyme Q 10 is a potent mitochondrial antioxidant, which also exhibits an antihypertensive effect⁴. Teran et al⁵ investigated the concentration of co-enzyme Q 10 in normal pregnancy and preeclampsia. The results demonstrated that during preeclampsia, there is a significant decrease in plasma levels of co-enzyme Q 10 compared with normal pregnant women and compared with those who are not pregnant.

### Lycopene

Lycopene is a dietary carotenoid with antioxidant activity. Yamini et al¹⁵ demonstrated that beta-carotene supplementation in pregnancy will increase its level in maternal serum. Sharma et al¹⁶ conducted a prospective clinical trial which randomized 251 primigravida women to receive oral lycopene (2 mg. twice daily beginning at 16-20 wks) or matched placebo. Women randomized to lycopene were less likely to develop preeclampsia (8.6% vs. 17.7%, P<0.05) than those who received placebo.

### Melatonin

Melatonin is a powerful scavenger of oxygen-free radicals and protects against oxidative mitochondrial damage in the placenta by inhibiting NADPH-dependent lipid peroxidation.⁶ Serum concentration of melatonin is reduced in women with severe preeclampsia compared with normotensive pregnant controls.⁷ When melatonin is administered to placental homogenates from late human gestation, there is a reduction in glutathione peroxidase activity, suggesting that melatonin supplementation may have direct placental antioxidant activity.⁸ In vitro studies suggest that melatonin protects against oxidative mitochondrial damage in an ischemia reperfusion model of preeclampsia.⁹

### Other Agents

Recently, Nitric Oxide has been shown to be endothelium derived relaxing factor (EDRF) from the endothelium of blood vessels. Nitric Oxide is considered to be a major mediator in the control of vascular reactivity. Nitric Oxide is synthesized from L-arginine by nitric oxide synthase located in endothelial cells. Nitric Oxide can also be generated by application of various nitric oxide donors such as Sodium Nitroprusside, Nitroglycerin, Glyceryl Trinitrite, SIN-1, Isosorbid Mononitrite, Isosorbid Dinitrite, etc.

Treatment of pregnant rats with nitric oxide synthase inhibitors, which are analogues of L-arginine (such as L-NAME, N.sup.G-nitro-L-arginine methyl ester) results in elevated blood pressure, fetal retarded growth and proteinuria. Thus, inhibition of Nitric Oxide synthesis produces conditions and symptoms identical to preeclampsia of pregnancy and establishes that preeclampsia is the direct result of the decrease in nitric oxide synthesis and/or a change in the regulation of vascular tone. These conditions give rise to increased blood pressure, decreased blood flow to the fetus, retarded fetal development and proteinuria. Agents which raise Nitric Oxide levels therefore are useful in the treatment of preeclampsia of pregnancy. Since Nitric Oxide donors also reduce contractility of the uterus during pregnancy, Nitric Oxide donors are also useful for use in preterm labor.

The nitric oxide effects on smooth muscle depend upon the activation of Guanylate Cyclase and generation of cGMP to produce relaxation and this step is progesterone dependent. Thus, combinations of Nitric Oxide donors with Progesterone are particularly efficacious for the treatment of preeclampsia and of preterm labor.

EP 0 441 119 A2 discloses the use of L-arginine in the treatment of hypertension and other vascular disorders. It suggests that the mechanism by which L-arginine is effective for this purpose is because it may be the physiological precursor of “the most powerful endothelial-derived releasing factor, Nitric Oxide.” The use of L-arginine in combination with other pharmaceutically active agents is not discussed in this publication.

### OBJECTS OF THE INVENTION

It is an object of this invention to provide a method for the prevention and treatment of preeclampsia and/or complications of maternal/placental oxidative stress with a combination of a vitamin agents, co-enzymes and amino acid donor.

It is another object to provide such a method in which this agent is used in combination with a cyclooxygenase inhibitor and/or donor for the prevention and treatment of preeclampsia, pregnancy-induced hypertension and/or intrauterine growth restriction, as well as for the treatment of complication relating to thrombophilia's, antiphospholipid syndrome all with a single pill.

A further object is the provision of pharmaceutical compositions useful in practicing the methods of this invention.

A further object is the provision of delivery of invention by oral, nasal, intravenous method of delivery by capsule, liquid, tablet, or nasal spray composition.

Other objects will be apparent to those skilled in the art to which this invention pertains.

### SUMMARY OF THE INVENTION

In a method aspect, this invention relates to a method of treating at least one of preeclampsia and preterm labor in a pregnant female which comprises administering to a pregnant female manifesting the symptoms thereof,
a) Thiamine and
b) Folic Acid (or active form 5-Methyl-Tetrahydrofolate i.e. >Metafolin®) and
c) Pyridoxine (vitamin B-6) or it active form Pyridoxine 5-phosphate (P5P)
d) Cyanocobalamin and
e) Ascorbic Acid and
f) Selenium and
g) Zinc and
h) N-acetyl Cysteine and
i) Co-enzyme Q10 and
j) Lycopene and/or
k) Melatonin and/or
l) Vitamin E and/or 81 mg Aspirin
   at least one of a cyclooxygenase inhibitor, a PGI.sub.2-mimetic, a thromboxane (TXA.sub.2) inhibitor, a compound possessing PGI.sub.2-agonistic and TXA.sub.2-inhibiting properties, a compound possessing TXA.sub.2-antagonistic and PGI.sub.2-mimetic activities, and a TXA.sub.2-antagonist, in amounts effective to ameliorate the symptoms of preeclampsia accompanied or unaccompanied by intrauterine growth restriction in a pregnant female mammal.

In another method aspect, this invention relates to a method of intrauterine growth restriction in a pregnant female which comprises administering to a pregnant female manifesting the symptoms thereof, amounts of
a) Thiamine and
b) Folic Acid (or active form 5-Methyl-Tetrahydrofolate i.e. >Metafolin®) and
c) Pyridoxine (vitamin B-6) or it active form Pyridoxine 5-phosphate (P5P)
d) Cyanocobalamin and
e) Ascorbic Acid and
f) Selenium and
g) Zinc and
h) N-acetyl Cysteine and
i) Co-enzyme Q10 and
j) Lycopene and/or
k) Melatonin and/or
l) Vitamin E and/or 81 mg Aspirin
   at least one of a cyclooxygenase inhibitor, a PGI.sub.2-mimetic, a thromboxane (TXA.sub.2) inhibitor, a compound possessing PGI.sub.2-agonistic and TXA.sub.2-inhibiting properties, a compound possessing TXA.sub.2-antagonistic and PGI.sub.2-mimetic activities, and a TXA.sub.2-antagonist, in amounts effective to ameliorate the symptoms of preeclampsia accompanied or unaccompanied by intrauterine growth restriction in a pregnant female mammal.

In a product aspect, this invention relates to a pharmaceutical composition comprising (a) a progestational agent and (b) at least one of a nitric oxide synthase substrate and a nitric oxide donor, alone or in further combination with one or more of a cyclooxygenase inhibitor, a PGI.sub.2-mimetic, a thromboxane (TXA.sub.2) inhibitor, a compound possessing TXA.sub.2-agonistic and TXA.sub.2-inhibiting properties, a compound possessing TXA.sub.2-antagonistic and PGI.sub.2-mimetic activities, and a TXA.sub.2 antagonist, with the amount of the progestational agent per unit dosage being bioequivalent to 50-300 mg. of injected progesterone and the amount of the nitric oxide synthase substrate, a nitric oxide donor or both per unit dosage being effective to, respectively, either raise the blood level of circulating L-arginine to at least about 1 mmole above the normally 2 to 3 mmolar circulating levels or raise the nitric oxide donor levels to about 1 to 1000 nmolar.

### DETAILED DISCLOSURE

The methods of this invention treat one or more of preeclampsia and preterm labor in a pregnant female mammal, preferably a human, who is manifesting the symptoms thereof or who is a high risk candidate for doing so, e.g., as determined by the progress of a present or previous pregnancy.

Because these abnormal conditions of pregnancy are produced by or aggravated by an increase in free radical production with depletion of antioxidants and substrate in the production of antioxidants both superoxide dismutase substrates, e.g., cysteine, glutathione and methione and B complex vitamins, e.g., thiamin, folic acid, Vitamin B6 (pryradoxime, active form Pryradoxine 5-phosphate), vitamin B12 (Cyanocobalamin) and vitamin antioxidant agents e.g., Ascorbic Acid, Selenium, Zinc, Copper, Vitamin E and Cyclooxygenase inhibitors are useful for ameliorating the symptoms thereof and, in one aspect of the method of this invention, a combination of both are employed.

A synergistic effect is achieved when the combination of agents is administered concurrently with a cyclooxygenase inhibitor.

Thus, the method aspect of this invention and the pharmaceutical composition aspect of this invention employs a combination of
a) Thiamine and
b) Folic Acid (or active form 5-Methyl-Tetrahydrofolate i.e. >Metafolin®) and
c) Pyridoxine (vitamin B-6) or it active form Pyridoxine 5-phosphate (P5P)
d) Cyanocobalamin and
e) Ascorbic Acid and
f) Selenium and
g) Zinc and
h) N-acetyl Cysteine and
i) Co-enzyme Q10 and
j) Lycopene and/or
k) Melatonin and/or
l) Vitamin E and/or 81 mg Aspirin at least one or more of a cyclooxygenase inhibitor, e.g., aspirin; a PGI.sub.2-mimetic, e.g., iloprost and cicaprost; a thromboxane (TXA.sub.2) inhibitor, e.g., dazoxiben hydrochloride (benzoic acid, 4-[2-(1H-imidazol-yl)ethoxy]-, monohydrochloride; UK 37248), dazmegrel (1H-indole-1-propanoic acid, 3-(1H-imidazol-1-ylmethyl)-2-methyl-; UK 3885), ozagrel (2-propenoic acid, 3-[4-(1-H-imidazol-1-ylmethyl)phenyl]-; OKY-046) and pirmagrel (imidazo[1,5-a)pyridine-5-hexanoic acid; CGS-13080); a compound possessing TXA.sub.2-agonistic and TXA.sub.2-inhibiting properties, e.g., ridogrel (pentanoic acid, 5-[[[3-pyridinyl[3-(trifluoromethyl)phenyl]methylene]-amino]oxy]-; R-68070) and labogrel (6-heptenoic acid, 7-phenyl-7-(3-pyridinyl)-; a compound possessing TXA.sub.2-antagonistic and PGI.sub.2-mimetic activities, e.g., 5-heptenoic acid, 7-[3-[[(diphenylmethoxy)-imino]-bicyclo,[2.2.1]hept-2-yl]-; EP 035-rac) and 5-heptenoic acid, 7-[3-[[(diphenylmethoxy)imino]methyl]biclo[2.2.2]-oct-5-en-2-yl]-(EP 157); and a TXA.sub.2 antagonist, e.g., 5-heptenoic acid, 7-[3-[[2-[(phenylamino)carbonyl]-hydrazino]methyl]7-oxabicyclo[2.2.1]hept-2-yl]-, 1S[1.alpha.,2.alpha.(Z),3.alpha.,4.alpha.]]-(SQ 29548); benzenepropanoic acid, 2-[[3-4[(pentylamino)carbonyl]-2-oxazolyl]-7-oxabicyclo[2.2.1]hept-2-ylmethyl}-(BMS 180291); acetic acid, [4-[2-[(phenylsulfonyl)amino]ethyl]phenoxy]-(sultroban, BM-13177); benzeneacetic acid, 4-[2-[[[4-chlorophenyl)sulfonyl]amino]ethyl]-(daltroban, BM-13505); (S-145 rac); 5-hexanoic acid, 6-[3-[[[(4-bromophenyl)sulfonyl]amino]methyl]bicyclo[2.2.1]hep-2-yl]-, decyl ester, [1S[1.alpha.2.alpha.2.alpha.(Z),-3.beta.,4.alpha.]]-(ONO 8809); 9H-carbazole-9-propanoic acid, 3-[[(4-fluorophenyl)sulfonyl]amino]-1,2,3,4-tetrahydro-, (R)-(bay-u-3405); and (4Z)-6-[(5S)-5-(4-chlorophenylsulfonyl(aminomethyl)-cycloent-1-enyl]4-hexanoic acid (ZU 154343).

Examples of combinations of active agents which can be administered concurrently with vitamin, coenzyme and amino acid formulation are low dose (e.g., 10-100 mg) of aspirin (or other cyclooxygenase inhibitor; PGI.sub.2-mimetics (e.g., iloprost, cicaprost); combinations of a PGI.sub.2-mimetic and low dose aspirin.

Examples of oral dosage ranges of Vitamin, coenzyme and amino acid substrate are:

| | total dose per day: Range |
|---|---|
| Thiamine | 500 mg–10 g |
| Pyradoxine (B6 or active form | 500–2000 μg/kg |
| Pyradoxim5-phosphate P5P) | |
| Folic acid (or active form | 0.5–10 mg |
| Metafolin ® at bioequivalent dose) | |
| Cyanocobalamin (B12) | 10–100 mg |
| Co-enzyme Q 10 | 10–100 mg |
| Selenium | 10–2000 μg |
| Zinc | 10–50 mg |
| Ascorbic Acid | 500–2000 mg |
| Lycopene | .1–4 mg |

The following are typical oral dosage ranges active agents of the vitamin E and the optional other active agents concurrently administered with the vitamin, coenzyme amino acid compound to enhance its performance.

Vitamin E: A daily dose bioequivalent to 100-800 IU. of vitamin E/day, e.g., an oral suspension or tablets or dragees providing an oral dose thereof of 100-1000 IU./day; or injectable solution of vitamin E daily with the bioequivalent of 100-1000 IU.

Melatonin: 10-20 mg/kg subcutaneous, parental dosing or oral dose bioequivalent.

Aspirin: 10-100 mg/kg/day oral dosing.

The pharmacologically active agents employed in this invention can be administered in admixture with conventional excipients, i.e., pharmaceutically acceptable liquid, semi-liquid or solid organic or inorganic carriers suitable, e.g., for parental or enteral application and which do not deleteriously react with the active compound in admixture therewith. Suitable pharmaceutically acceptable carriers include but are not limited to water, salt solutions, alcohols, vegetable oils, polyethylene glycols, gelatin, lactose, amylose, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds.

For parental application, particularly suitable are solutions, preferably oily or aqueous solutions, as well as suspensions, emulsions, or implants, including suppositories. Ampules are convenient unit dosages.

In a preferred aspect, the composition of this invention is adapted for ingestion.

For enteral application, particularly suitable are unit dosage forms, e.g., tablets, dragees or capsules having talc and/or a carbohydrate carrier or binder or the like, the carrier preferably being lactose and/or corn starch and/or potato starch; particulate solids, e.g., granules; and liquids and semi-liquids, e.g., syrups and elixirs or the like, wherein a sweetened vehicle is employed. Sustained release compositions can be formulated including those wherein the active compound is protected with differentially degradable coatings, e.g., by microencapsulation, multiple coatings, etc.

Suitable for oral administration are, inter alia, tablets, dragees, capsules, pills, granules, suspensions and solutions. Each unit dose, e.g., each tablespoon of liquid or each tablet, or dragee contains, for example, 5-5000 mg. of each active agent.

Solutions for parenteral administration contain, for example, 0.01-1% of each active agent in an aqueous or alcoholic solution.

The combination of active agents is preferably administered at least once daily (unless administered in a dosage form which delivers the active agents continuously). Since the LD.sub.50 dosages of most of these active agents is known in the prior art, a lower dosage regimen can be initiated and the dosage increased until a positive effect is achieved or a higher dosage regimen can initially be employed, e.g., in a crisis situation, and the dosages regulated downward as relief from the symptoms is achieved.

Without further elaboration, it is believed that one skilled in the art can, using the preceding description, utilize the present invention to its fullest extent. The preferred specific embodiments are, therefore, to be construed as merely illustrative, and not limitative of the disclosure in any way whatsoever.

The entire disclosure of all applications, patents and publications, cited above and below are hereby incorporated by reference.

It can be concluded from these studies that the combination of the nutritional/vitamin compound provides results which are synergistic and may not be achieved with a single nutritional vitamin, co-enzyme or amino acid alone. These studies show that the basis for this effectiveness lies in the ability of the each agent to increase the effectiveness of the antioxidant system to decrease free radicals and lipidperoxidation. Thereby lower blood pressure as well as improving fetalmaternal profusion, thereby increasing fetal weight.

The combined effect of the combination of these agents is surprisingly dramatic and, more importantly, the significant fetal and maternal effects observed with treatment with the combination. Prior medical evidence does not suggest that the combination would provide these advantages, because the basis for them is not the simple combination of two agonistic compounds but instead is the synergistic effect on the immune system ability to reduce oxidative stress.

The method of treatment employed in this invention can also be employed for the treatment of sepsis, pneumonia, hypoxic injury, post surgical, or other condition resulting in depletion of antioxidants (in both females and males).

Without further elaboration, it is believed that one skilled in the art can, using the preceding description, utilize the present invention to its fullest extent. The preferred specific embodiments are, therefore, to be construed as merely illustrative, and not limitative, of the disclosure in any way whatsoever.

The entire disclosure of all applications, patents and publications, cited above and below, are hereby incorporated by reference.

### EXAMPLES

### Example 1

### Treatment of Preeclampsia

To a pregnant human female (ca 20-40 years; 60-80 kg.) usually in her second half of pregnancy and displaying the symptoms of preeclampsia, including hypertension (above 140 mm. systolic and above 90 mm.h diastolic), edema and proteinuria, administration of the vitamin/nutritional compound daily may reduce the severity or stop the progression of the disease.

### Example 2

### Prevention of Preeclampsia

To a pregnant human female comparable to one described in Example 1, who has risk factors for preeclampsia i.e.; abnormal ultrasound findings—elevated uterine artery Doppler indices and/or “notching”, administration of daily administration of the vitamin/nutritional compound daily may reduce the severity of disease and/or stop the progression of the disease thereby allowing for a longer gestation by delaying the onset of preeclampsia. This will result in a reduction of neonatal morbidity and mortality associated with preterm birth.

### Example 3

### Prevention of Preeclampsia with Inherited Genetic Conditions

To a pregnant human female comparable to one described in Example 1, who has a genetically inherited thombophilia i.e.; MTHFR deficiency, Factor V leiden, prothrombin 20210 mutation, folate receptor autoantibodies or other inheritable condition resulting in an increased risk of placental thrombosis and subsequent placenta hypoxia and/or increased oxidative free radical production, and/or lipid peroxidase, daily administration of the vitamin/nutritional compound daily may reduce the severity of disease and/or stop the progression of the disease thereby allowing for a longer gestation by delaying the onset of preeclampsia. This will result in a reduction of neonatal morbidity and mortality associated with preterm birth.

### Example 4

### Prevention of Preeclampsia with Medical Risk Factors

To a pregnant human female comparable to one described in Example 1, who has underlying medical condition leading to an increased risk for developing preeclampsia i.e.; underlying vascular disease, renal disease, autoimmune disease, diabetes, cardiopulmonary disease, infection or other process leading to an increased oxidative stress load. Administration daily of the vitamin/nutritional compound daily may reduce the severity of disease and/or stop the progression of the disease thereby allowing for a longer gestation by delaying the onset of preeclampsia. This will result in a reduction of neonatal morbidity and mortality associated with preterm birth.

### Example 5

Use in decreasing inflammation as a result of free radical oxidation and lipid peroxidase. To a pregnant human female comparable to one described in Example 1, who has preterm labor due to the fetal inflammatory response or premature rupture of the membranes. The nutritional/vitamin compound may be utilized with current treatments of antibiotics and tocolytic agents. The addition of the compound may result in further prolongation of the pregnancy by decreasing inflammatory mechanism of preterm labor.

### Example 6

In a human (male or female) who is critical ill or post operative from surgery, when there is suspected hypoxic/reprofusion injury. Administration daily of the vitamin/nutritional compound daily may aid in reducing the severity of disease and/or stop the progression of the disease thereby decreasing end organ damage.

The preceding examples can be repeated with similar success by substituting the generically or specifically described reactants and/or operating conditions of this invention for those used in the preceding examples.

From the foregoing description, one skilled in the art can easily ascertain the essential characteristics of this invention, and without departing from the spirit and scope thereof, can make various changes and modifications of the invention to adapt it to various usages and conditions.

### REFERENCES

¹ Saeed S A, Mitchell M D. Conversion of arachidonic acid to lipoxygenase products by human fetal tissues. Biochem Med. 1983; 30:322-327.
² Thakur S, Gupta N, Kakkar P. Serum copper and zinc concentrations and their relation to superoxide dismutase in severe malnutrition. Eur J. Pediatr. 2004; 163(12):742-4.
³ Barua R S, Ambrose J A, Srivastava S, et al. Reactive oxygen species are involved in smoking-induced dysfunction of nitric oxide biosynthesis and upregulation of endothelial nitric oxide synthase: an in vitro demonstration in human coronary artery endothelial cells. Circulation. 2003; 107:2342-2347.
⁴ Burke B E, Neuenschwander R, Olson R D. Randomized, double-blind, placebo-controlled trial of coenzyme Q10 in isolated systolic hypertension. South Med J. 2001; 94:1112-1117.
⁵ Teran E, Racines-Orbe M, Vivero S, et al. Preeclampsia is associated with a decrease in plasma coenzyme Q10 levels. Free Radic Biol Med. 2003; 35:1453-1456.
⁶ Milczarek R, Klimek J, Zelewski L. Melatonin inhibits NADPH-dependent lipid peroxidation in human placental mitochondria. Horm Metab Res. 2000; 32:84-85.
⁷ Nakamura Y, Tamura H, Kashida S, et al. Changes of serum melatonin level and its relationship to feto-placental unit during pregnancy. J Pineal Res. 2001; 30:29-33.
⁸ Okatani Y, Wakatsuki A, Shinohara K, et al. Melatonin stimulates glutathione peroxidase activity in human chorion. J Pineal Res. 2001; 30:199-205.
⁹ Okatani Y, Wakatsuki A, Shinohara K, et al. Melatonin protects against oxidative mitochondrial damage induced in rat placenta by ischemia and reperfusion. J Pineal Res. 2001; 31:173-178.
¹⁰ Kupfermine M J. Thrombophilia and pregnancy. Reprod Biol Endocrinol. 2003; 1:11-133.
¹¹ Chappell L C, Seed P T, Briley A L, et al. Effect of antioxidants on the occurrence of preeclampsia in women at risk: a randomized trial. Lancet. 1999; 354:810-816.
¹² Rayman M P, Abou-Shakra F R, Ward N I, et al. Comparison of selenium levels in preeclamptic and normal pregnancies. Biol Trace Elem Res. 1996; 55:9-20.
¹³ Rayman M P, Bode P, Redman C W. Low Selenium status is associated with the occurrence of the pregnancy disease preeclarnpsia in women from the United Kingdom. Am J Obstet Gynecol. 2003; 189:1343-1349.
¹⁴ Han L, Zhou S M. Selenium supplement in the prevention of pregnancy-induced hypertension. Chin Med J (Engl). 1994; 107:870-871.
¹⁵ Yamini S, West K P Jr, Wu L, et al. Circulating levels of retinol, tocopherol and carotenoid in Nepali pregnant and postpartum women following long-term beta-carotene and vitamin A supplementation. Eur J Clin Nutr. 2001; 55:252-259.
¹⁶ Sharma J B, Kumar A, Malhotra M, et al. Effect of lycopene on pre-eclampsia and intra-uterine growth retardation in primigravidas. Int J Gynaecol Obstet. 2003; 81:257-262.

What is claimed is:

1. A pharmaceutical composition comprising an admixture of effective amounts of:
Thiamine and
(b) Folic Acid (or active form 5-Methyl-Tetrahydrofolate i.e. >Metafolin®) and
(c) Pyridoxine (vitamin B-6) or it active form Pyridoxine 5-phosphate (P5P)
(d) Cyanocobalamin and
(e) Ascorbic Acid and
(f) Selenium and
(g) Zinc and
(h) N-acetyl Cysteine and
(i) Co-enzyme Q10 and
(j) Lycopene and/or
(k) and/or Melatonin
(l) Vitamin E and/or 81 mg Aspirin
at least one of a cyclooxygenase inhibitor, a PGI.sub.2-mimetic, a thromboxane (TXA.sub.2) inhibitor, a compound possessing PGI.sub.2-agonistic and TXA.sub.2-inhibiting properties, a compound possessing TXA.sub.2-antagonistic and PGI.sub.2-mimetic activities, and a TXA.sub.2-antagonist, in amounts effective to ameliorate the symptoms of preeclampsia accompanied or unaccompanied by intrauterine growth restriction in a pregnant female mammal.

2. The composition according to claim 1, wherein (b-l) are important in the in vivo production of antioxidant or in the reduction of oxidants, lipid peroxidase, myeloperoxidase, homocysteine, MDA, or other in vivo oxidative, thrombogenic substrate or substance.

3. The composition according to claim 1, wherein (h) amino acid donor in the formation of glutathione in vivo.

4. The composition according to claim 1, which (l) comprises a cyclooxygenase inhibitor.
